(19) United States
(12) Patent Application Publication
(10) Pub. No.: US 2020/0345685 A1
(43) Pub. Date: Nov. 5, 2020

(54) TOPICAL APPLICATION OF MEDICAL CANNABIS

(71) Applicant: Christopher Ayo Otiko, Tarzana, CA (US)

(72) Inventor: Christopher Ayo Otiko, Tarzana, CA (US)

(21) Appl. No.: 16/401,559

(22) Filed: May 2, 2019

(51) Int. Cl. A61K 31/352; A61K 9/00; A61K 31/10; A61K 31/08; A61K 31/593; A61K 9/70; A61K 31/05; A61K 47/22

(52) U.S. Cl. CPC A61K 31/352; A61K 9/0014; A61K 31/10; A61K 47/22; A61K 31/593; A61K 9/7023; A61K 31/05; A61K 31/08

(57) ABSTRACT

A medicinal cannabis mixture has cannabis oil and one or more transdermal agents that increase permeability of skin to passage of cannabis. In a process of administering the mixture the solution is applied to a subject's skin over an artery.

### CROSS-REFERENCE TO RELATED APPLICATIONS

N/A

### BACKGROUND OF THE INVENTION

### 1. Field of the Invention

The present invention is in the technical field of procedures for administering medical cannabis through the skin of a person.

### 2. Description of Related Art

Methods and products for transdermally administering particular chemicals are well-known in the art. Several U.S. patents have issued for transdermal application of chemicals, for example nicotine. To the inventor's knowledge, however, particular processes for introducing cannabis-based medications into the bloodstream of a subject have not been developed and proven. What is clearly needed are formulations and processes to accomplish a very efficient introduction of cannabis-based medicines into the bloodstream.

### BRIEF SUMMARY OF THE INVENTION

In an embodiment of the invention a medicinal cannabis mixture is provided, comprising cannabis oil and one or more transdermal agents that increase permeability of skin to passage of cannabis.

In one embodiment the one or more transdermal agents comprise one of dimethyl sulfoxide (DMSO) and Dipropylene glycol (DPG). Also, in one embodiment the one or more transdermal agents comprise both dimethyl sulfoxide (DMSO) and Dipropylene glycol (DPG). In one embodiment the mixture further comprises ascorbic acid as a stabilizing agent.

In one embodiment the medicinal cannabis mixture further comprises cholecalciferol (Vitamin D). Also in one embodiment the cannabis oil comprises one or more of tetranydrocannabinol (THC), cannabinol (CB), cannabidiol (CBD) and cannabichromene (CBC).

In another aspect of the invention a process for administering medicinal cannabis to a subject is provided, comprising preparing a mixture of cannabis oil and one or more transdermal agents that increase permeability of skin to passage of cannabis, and applying the mixture to a subject's skin directly over either a carotid artery in the subject's neck, or directly over either or both of an ulnar or a radial artery in the subjects wrist.

In one embodiment the mixture of cannabis oil and one or more transdermal agents is administered by one or more drops from a syringe. Also in one embodiment the mixture of cannabis oil and one or more transdermal agents is administered by transfer from a cotton swab carrying the mixture of cannabis oil and the one or more transdermal agents. And in one embodiment the mixture of cannabis oil and one or more transdermal agents is administered by application to a patch or bandage, which is then affixed to the skin of the subject.

In one embodiment of the process the one or more transdermal agents comprise one of dimethyl sulfoxide (DMSO) and Dipropylene glycol (DPG). In one embodiment the one or more transdermal agents comprise both dimethyl sulfoxide (DMSO) and Dipropylene glycol (DPG). In one embodiment the process further comprises ascorbic acid as a stabilizing agent. In one embodiment the process further comprises cholecalciferol (Vitamin D). And in one embodiment the cannabis oil comprises one or more of tetranydrocannabinol (THC), cannabinol (CB), cannabidiol (CBD) and cannabichromene (CBC).

### BRIEF DESCRIPTION OF THE SEVERAL VIEWS OF THE DRAWINGS

FIG. 1 is a flow chart depicting steps in a process according to an embodiment of the invention.

FIG. 2A is a diagrammatical view of location of regions for topical application in a subject's neck, in an embodiment of the invention.

FIG. 2B is a diagrammatical view of location of regions for topical application in a subject's wrist, in an embodiment of the invention.

FIG. 3 is a graph depicting limited-source diffusion in an embodiment of the invention.

FIG. 4 is a graph depicting CBD concentration v. depth in cm. for application via topical solution after 10 seconds.

FIG. 5 is a graph depicting CBD concentration v. depth in cm. for application via topical solution after 60 seconds.

FIG. 6 is a graph depicting CBD concentration v. depth in cm. for application via topical solution after 8 hours.

FIG. 7 is a graph depicting CBD concentration v. depth in cm. for application via topical solution after 24 hours.

FIG. 8 is a graph depicting CBD concentration v. depth in cm. for application via ointment after 10 minutes.

FIG. 9 is a graph depicting CBD concentration v. depth in cm. for application via ointment after 1 hour.

FIG. 10 is a graph depicting CBD concentration v. depth in cm. for application via ointment after 8 hours.

FIG. 11 is a graph depicting CBD concentration v. depth in cm. for application via ointment after 24 hours.

### DETAILED DESCRIPTION OF THE INVENTION

Medical marijuana is recognized and accepted at the time of this application in most medical and social contexts. In one circumstance the senior population has turned more and more to medical marijuana for relief from the pains and chronic troubles of an aging anatomy. One drawback for many is that the principle process for incorporating medical marijuana products into the person is through smoking the burning marijuana leaf, or through breathing vapors of marijuana oil, vaporized in a variety of ways. These are processes to which not everyone is partial. Far fewer people smoke now as once did, and the dangers of vapor and smoke inhalation are well-known.

Oral ingestion is a valid alternative to smoke and vapor, but there are problems as well with the somewhat longer process of ingestion and digestion required in this process.

The present inventor has developed and tested formulations and processes for efficiently introducing medical cannabis products directly into a subject's bloodstream through the skin, that is, transdermally, particularly proximate the carotid artery and arteries in the wrists of both hands.

In one embodiment of the present invention medicinal cannabis is delivered directly to a subject's bloodstream. The topical mixture and the process, and alternative embodiments, are described below in enabling detail.

### Cannabis Material

The point and purpose of the invention is to deliver cannabis into a subject's bloodstream more efficiently and in less time than can be done in prior art methods and with prior art materials.

In embodiments of the present invention a cannabis solution is preferably extracted from a natural source, such as marijuana or hashish, or any one or more compounds or chemical components of marijuana or hashish, including tetranydrocannabinol (THC), cannabinol (CB), cannabidiol (CBD) and cannabichromene (CBC).

Characteristics of a typical cannabis material useful with the invention are:
1) Solubility of about 2 microgram/ml in water at 37 C.
2) Solubility of about 500 milligram/ml or greater in light mineral oil at 37 C.
3) Molecular weight ranges from about 300 to 350 gm/mol.

It is to be noted that these characteristics are not limiting, but preferable, and may vary in alternative embodiments.

### Transdermal Agents

The transdermal agents preferred in embodiments of the invention are Dimethyl Sulfoxide (DMSO), and Dipropylene Glycol (DPG). These are two agents that allow transdermal delivery of cannabis into the bloodstream across the barrier of the subject's skin. In some embodiments one or the other of these two transdermal agents may be used. Preferably the two are both present in the inventive process, and the inventor has reason to believe that the two together provide a synergistic effect. Other examples of suitable carriers include ionically-charged materials, such as urea, which polarize the skin's molecules and increase the skin's permeability through ionic force.

### Other Ingredients

In some embodiments of the invention additional ingredients are included, primarily to make the resulting solution stable, which may include cholecalciferol (vitamin D) and ascorbic acid.

### The Process

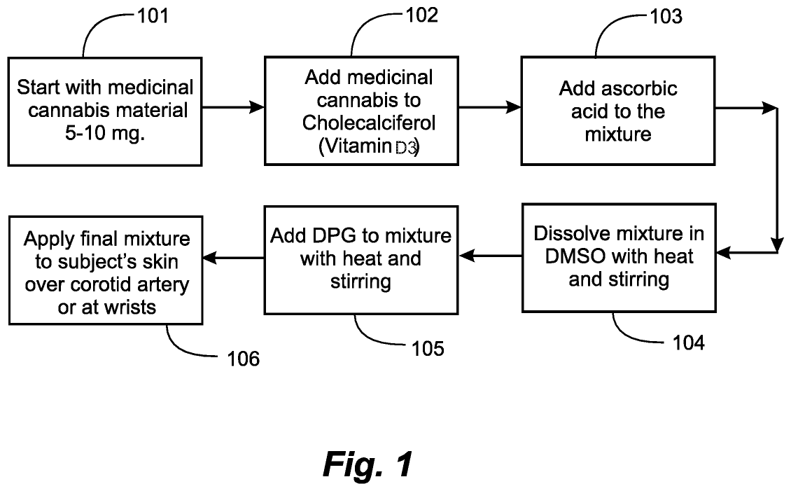

FIG. 1 is a flow chart illustrating steps in an overall process of preparing and administering medicinal cannabis through transdermal application. The process starts at step 101 with medicinal cannabis material, in one embodiment with an amount of about 5 to 10 mg. The material may be any one of many medicinal cannabis formulations, as described above. At step 102 the medicinal cannabis material is added to a solution of cholecalciferol (Vitamin D). The concentration and amount of Vitamin D may vary widely in different embodiments.

At step 103 ascorbic acid, in one embodiment in a concentration of from 0.1% to about 3% is added to the mixture as a stabilizer. The overall concentration of the stabilizer may be from 3% to 10%.

At step 104 the mixture is dissolved in dimethyl sulfoxide (DMSO), in concentration of from 5% to 20%, with heat and stirring. DMSO is an agent that acts as a chemical carrier that increases the permeability of the user's skin with respect to cannabis. The main ingredient of cannabis is THC, which is known, due to its high lipophilicity, to exhibit strong tendency to bind to tissue and protein, making transdermal application difficult. Fatal misuse has also been a concern in previous transdermal applications, but cannabinoids are rarely fatal when overdosed.

At step 105 dipropylene glycol (DPG) is added to the mixture with heat and stirring as well. DPG is another agent that is known to increase permeability of a subject's skin, and is believed by the inventor to work in a synergistic relationship with DMSO. Oleic acid may also be implemented in a synergistic manner with DMSO and DPG either singly or in combination to enhance permeability of the skin allowing the cannabis material to be absorbed.

At step 106 the resulting mixture is applied to a subject's skin over either or both of the subject's carotid arteries at the neck, or over arteries at the subject's wrists. The application of the unique mixture may be done in a number of ways. In one embodiment the final mixture may be drawn into a small syringe, alternatively a cotton swab may be used, dipped in the mixture, and then applied to the subject's skin. The mixture may also be applied to a patch or bandage, which may be applied, for example, by peripheral adhesive to the skin.

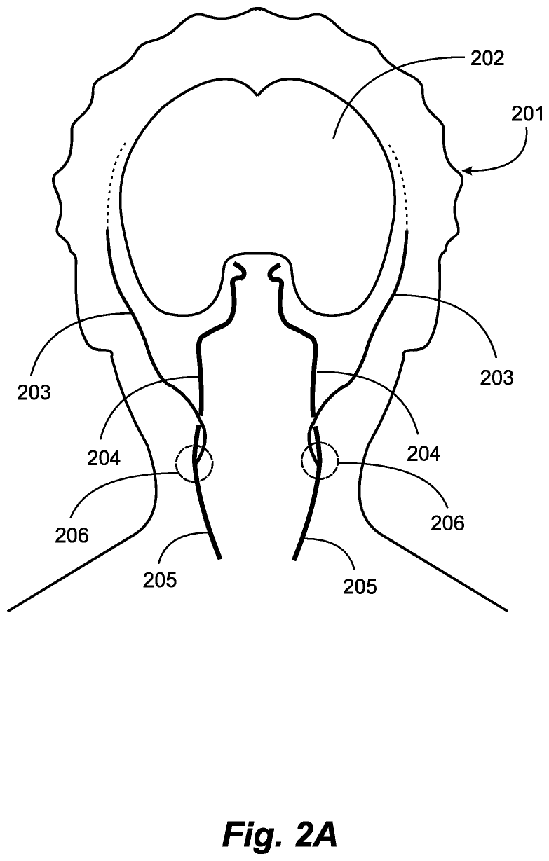

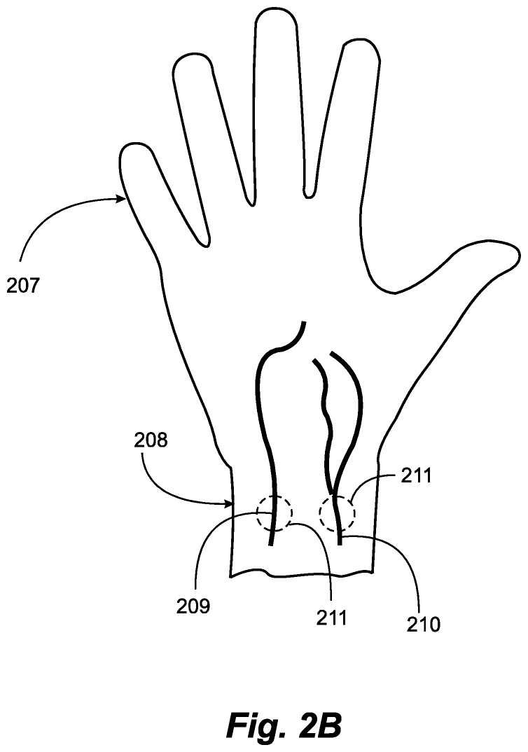

FIGS. 2A and 2B are diagrams depicting the preferable regions of application for a subject. FIG. 2A illustrates an outline 201 of a subject's head and shoulders. The subject's brain 202 is fed by blood through twin internal and external carotid arteries labeled as elements 204 and 203. These proceed from a common carotid artery 205 on each side. Regions 206, where the common branches to the internal and the external are preferable places for topical application on the skin at the neck.

FIG. 2B illustrates an outline 207 of a subject's hand, with the wrist region 208 and the approximate location in the wrist of the ulnar artery 209 and the radial artery 210. Regions 211 on the skin directly over the radial and the ulnar artery are preferable regions for topical application in embodiments of the present invention.

In various applications the inventor has found that, with application at the preferable points shown in FIGS. 2A and 2B the cannabis is transported into the bloodstream within 5 minutes. The therapeutic effects last for 4-6 hours.

In an alternative embodiment, the medical cannabis material is mixed as described with one or both of vitamin D and ascorbic acid, but without the transdermal agents DMSO and DPG, which may be separately applied to the skin over the preferable arterial points prior to the application of the medical cannabis material.

Additionally, the present inventor has discovered that oleic acid vesicles are very effective carriers for enhancing the penetration of drug molecules through the stratum corneum of the skin with the reduction of toxicity. Oleic acid vesicles are cheaper in cost and their method of preparation is simple. Prepared oleic acid vesicles are spherical in shape and of size below 500 nm. These vesicles are enabled to entrap the cannabis material for absorption into the skin and may be used in conjunction with DMSO and DPG, wherein in combination enhance the transdermal effect of the cannabis solution. Some time may be allowed to elapse after the application of the transdermal agent or agents, and before the application of the medical cannabis material, to allow the transdermal agents to accomplish the purpose.

In various circumstances the formulations of the invention, and the procedures for administering same for absorption into a subject's blood stream may be used for (1) treatment of nausea and pain associated with cancer and chemotherapy; (2) nausea, pain and wasting associated with AIDS; (3) arthritis and rheumatism; (4) glaucoma; (5) migraines; (6) muscle spasticity associated with multiple sclerosis and paralysis; (7) alcohol and narcotics withdrawal; (8) stress and depression; (9) asthma; and (10) epileptic seizures.

### Diffusion of CBD Through Skin and Underlying Tissue, When Applied Topically

The inventors in developing the materials and procedures basic to the instant invention have also calculated absorption of CBD materials into human flesh in terms of concentration at depth over time for application both by topical solution and by ointment.

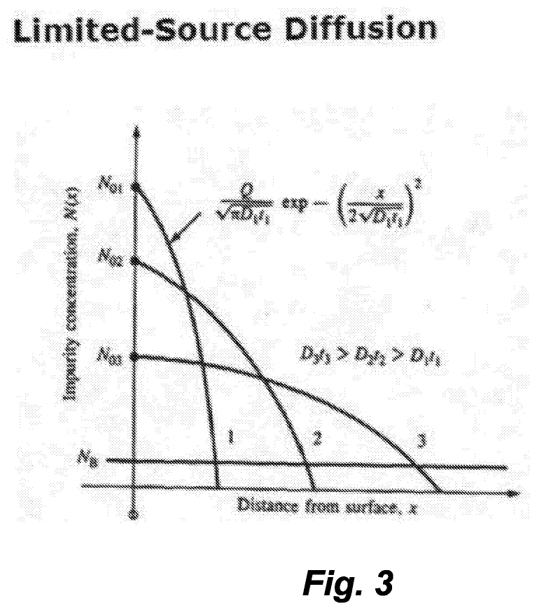

In these calculations following assumptions apply:
1. A thin application (topical dose) is applied and rubbed in. This would correspond to a limited source diffusion of CBD through the stratum corneum and into the underlying tissue.
2. The diffusion coefficient for both the stratum corneum and the underlying tissue is the same value. D=1.66×10−5 cm2/sec is taken as a reasonable value for transdermal penetration of human skin, both through the stratum corneum and the underlying tissues.
FIG. 3 is a graph showing relationships for limited-source diffusion.

A gaussian distribution results from a limited-source diffusion or constant total dopant diffusion. As the Dt product increases, the diffusion front moves more deeply into the skin and underlying tissue, and the surface concentration decreases. The area under each of the three curves in FIG. 3 is the same.

Limited-source diffusion is modeled mathematically using an impulse function at the skin surface as the initial boundary condition. The magnitude of the impulse is equal to the dose Q.

$$Q = 2 {N}_{0} \sqrt{Dt / π} = {\int }_{0}^{∞} N ( x , t ) dx$$

Initial condition

At t=0, N(x, 0)=0

Boundary conditions

$${\int }_{0}^{∞} N ( x , t ) dx = Q$$

$$And C ( ∞ , t ) = 0$$

The solution of Ficks second law that satisfies the initial and boundary conditions is given by:

$$N ( x , t ) = \frac{Q}{\sqrt{π Dt}} exp - {( \frac{π}{2 \sqrt{Dt}} )}^{2}$$

By setting x=0 we obtain the surface concentration

$${N}_{0} = N ( 0 , t ) = \frac{Q}{\sqrt{π Dt}}$$

The solution can be rewritten as

$$N ( x , t ) = {N}_{0} exp - {( \frac{π}{2 \sqrt{Dt}} )}^{2}$$

If N/NO=1 this is the equation for constant source diffusion.

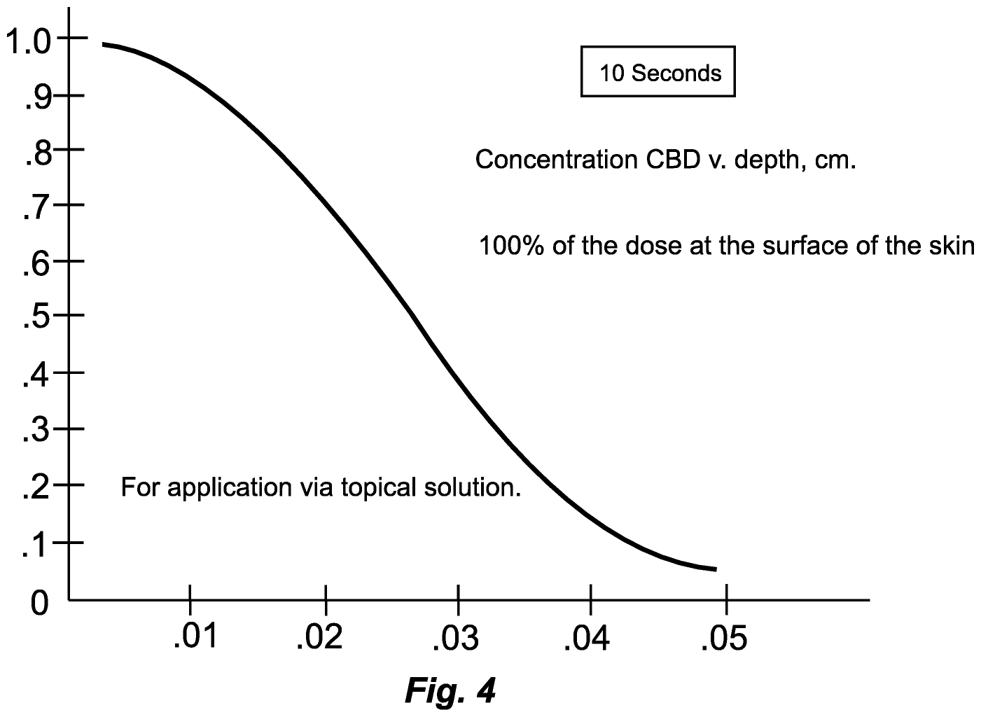

This diffusion theory was used to calculate the concentration vs. depth of CBD through human skin, using the diffusion constant, D, as determined experimentally. FIGS. 4-11 are results of these calculations. A liquid solution is assumed to be a thin topical application, usually rubbed into the skin, and therefore capable of providing a limited dose from the thin film.

FIG. 4 is a graph depicting CBD concentration v. depth in cm. for application via topical solution after 10 seconds.

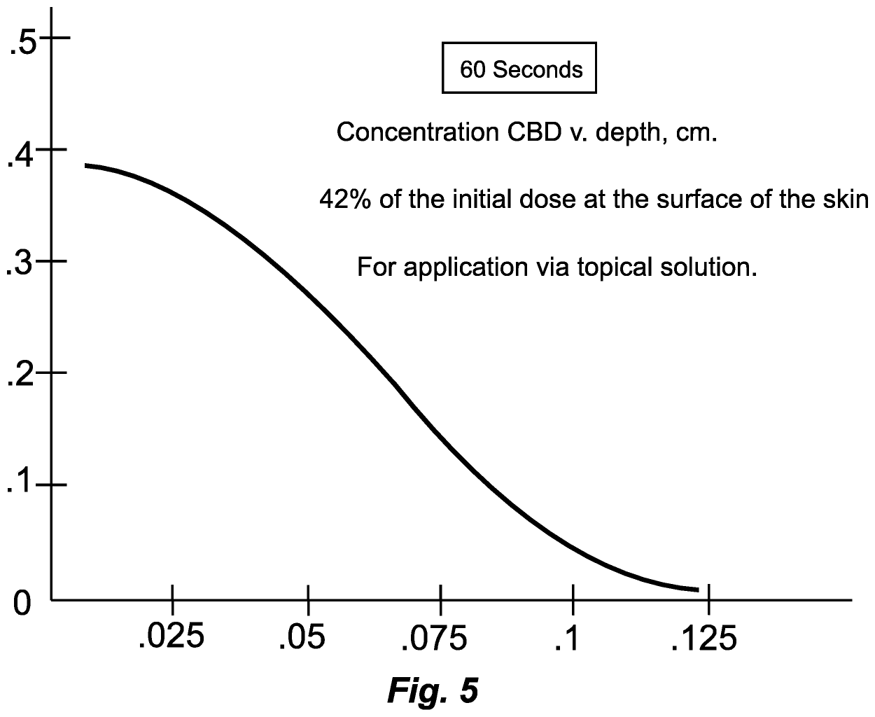

FIG. 5 is a graph depicting CBD concentration v. depth in cm. for application via topical solution after 60 seconds.

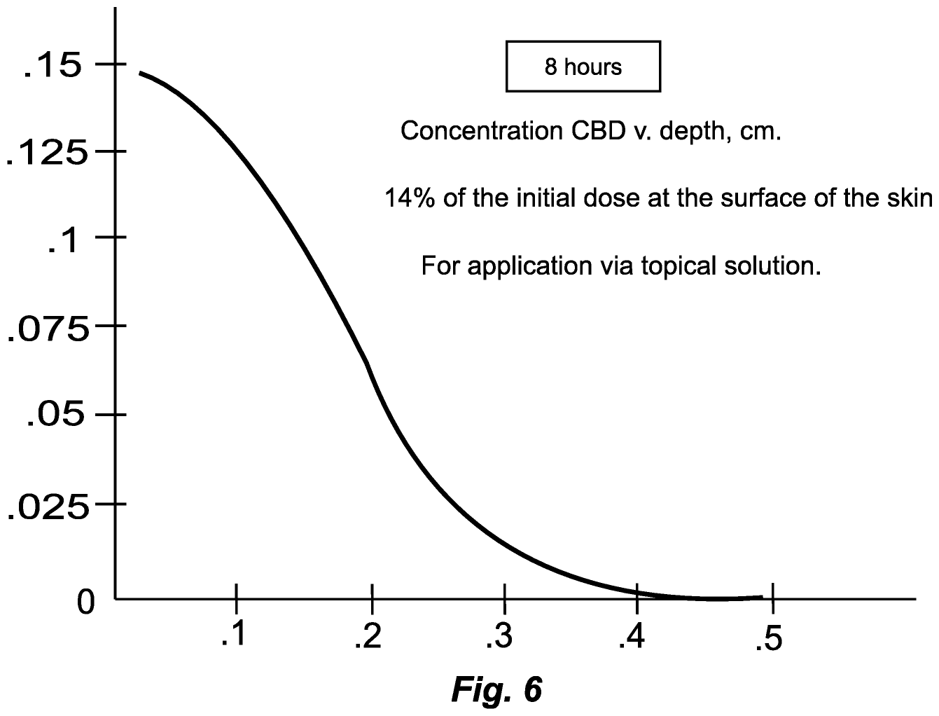

FIG. 6 is a graph depicting CBD concentration v. depth in cm. for application via topical solution after 8 hours.

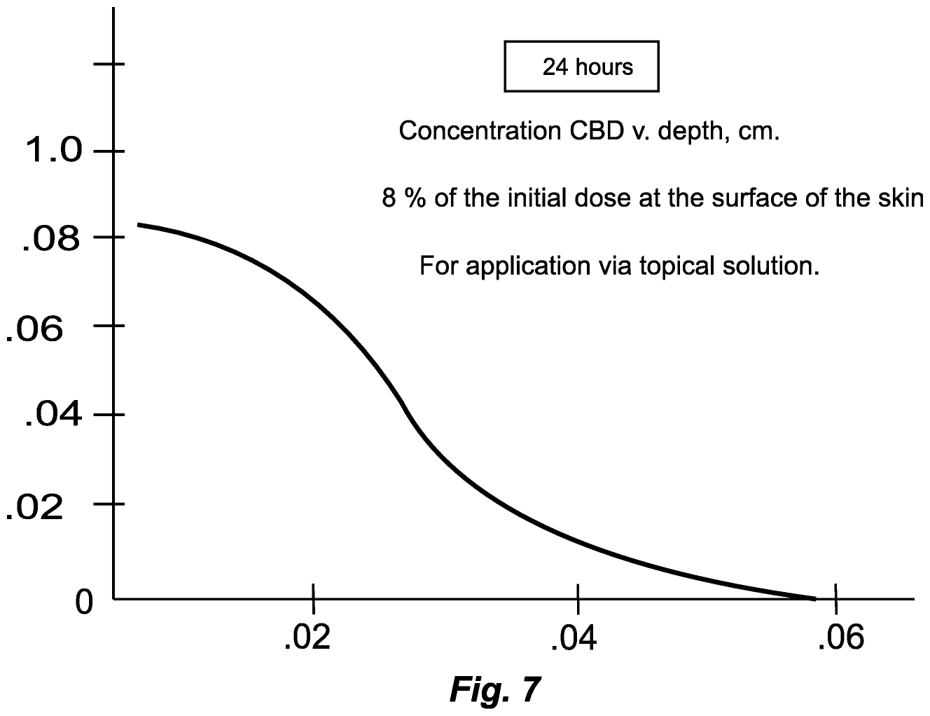

FIG. 7 is a graph depicting CBD concentration v. depth in cm. for application via topical solution after 24 hours.

The area under the curve is the same value in FIGS. 4-7, which are calculated for topical solution application. This is because the dose (total number of tetracycline molecules) remains the same, but is distributed deeper in the tissue, for a limited-source diffusion.

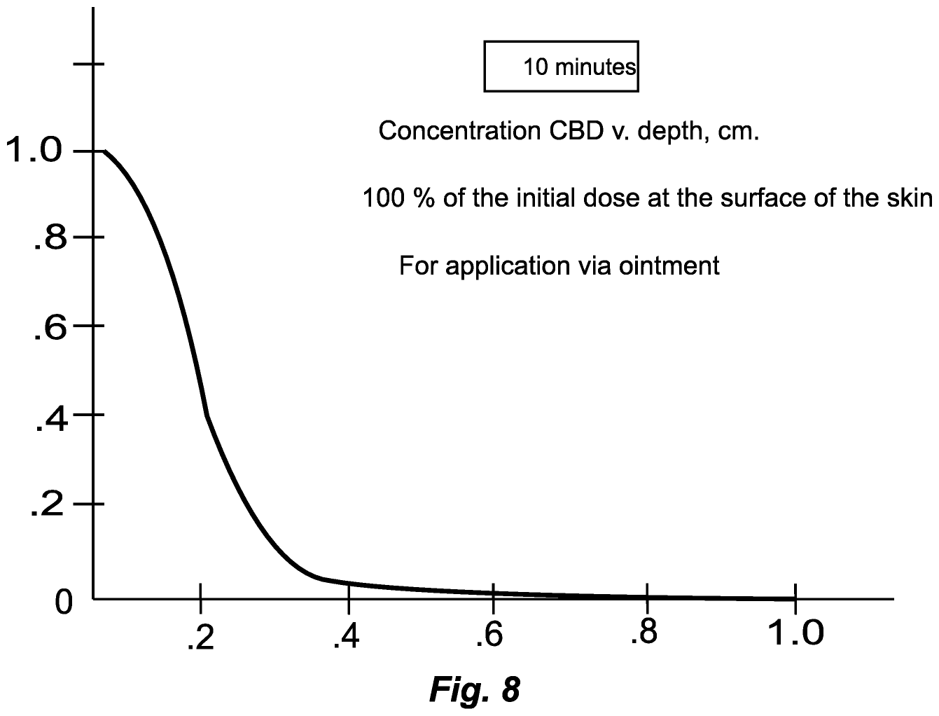

An ointment is assumed to be a thick topical application, not rubbed into the skin, and therefore capable of providing a constant dose from the thick film. FIGS. 8-11 distributions are calculated for a constant source diffusion of CBD when delivered in the form of an ointment.

FIG. 8 is a graph depicting CBD concentration v. depth in cm. for application via ointment after 10 minutes.

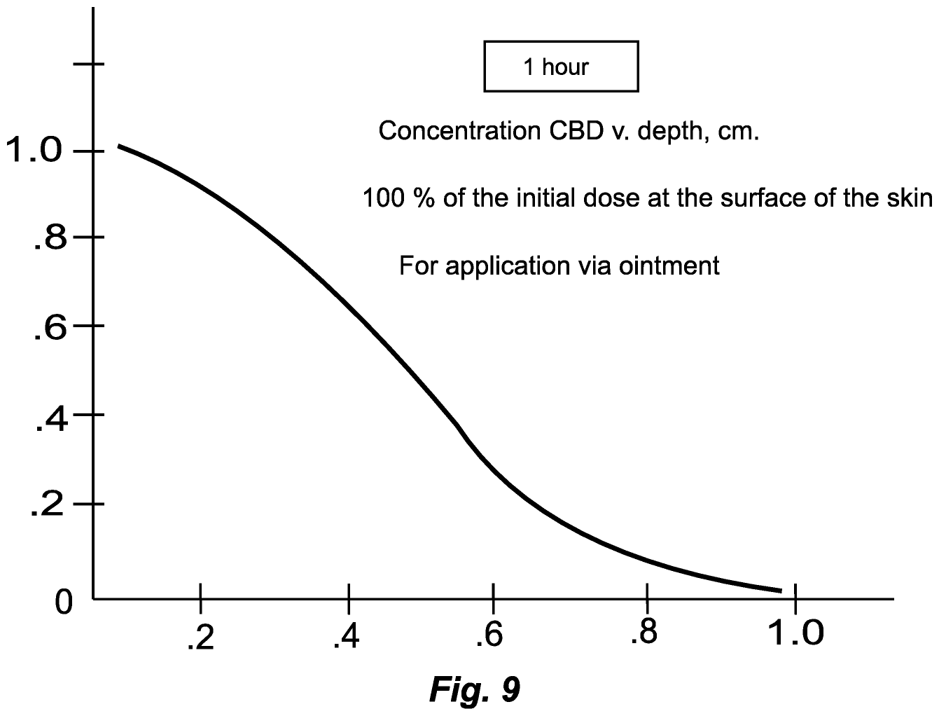

FIG. 9 is a graph depicting CBD concentration v. depth in cm. for application via ointment after 1 hour.

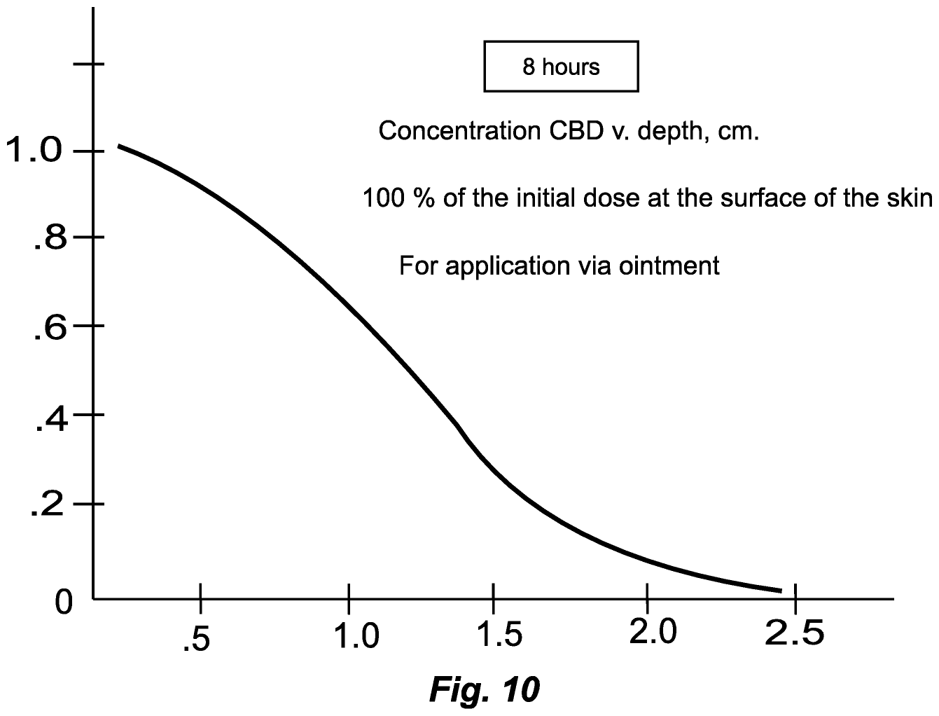

FIG. 10 is a graph depicting CBD concentration v. depth in cm. for application via ointment after 8 hours.

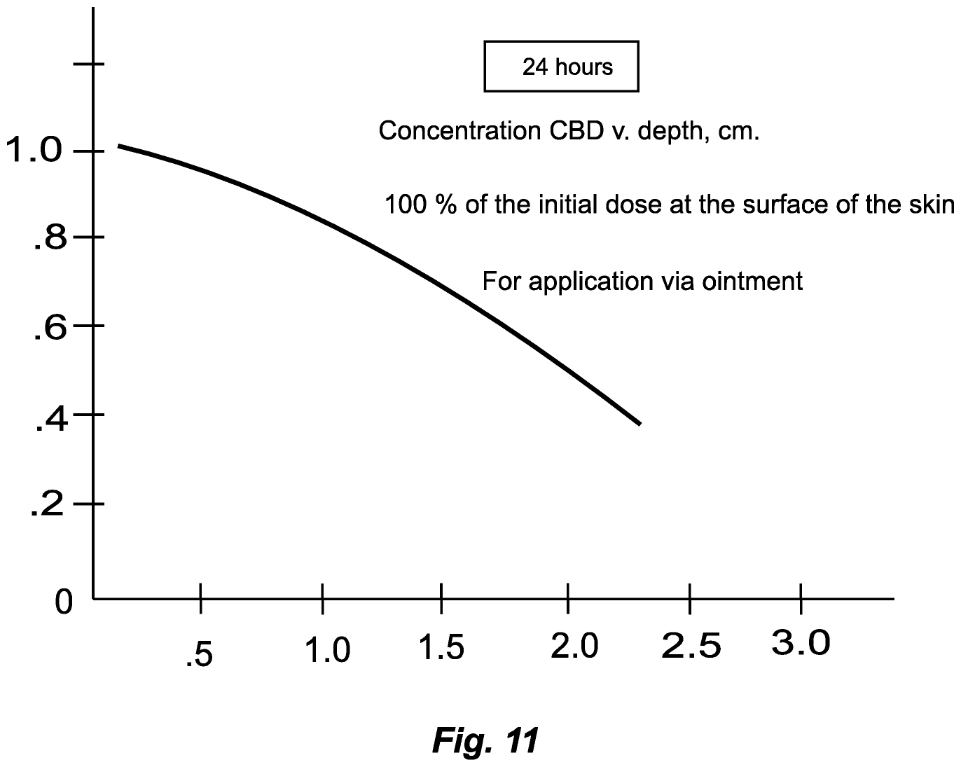

FIG. 11 is a graph depicting CBD concentration v. depth in cm. for application via ointment after 24 hours.

Conclusion: A higher dose at a deeper tissue penetration results from an ointment method of delivery, as compared to a solution method of delivery, as may be seen from the graphs of FIGS. 4-11.

It will be apparent to the skilled person that the formulations and procedures described above in various embodiment of the invention are exemplary, and not limiting. The formulations and procedures may be modified in many ways within the scope of the invention. The scope of the invention is limited only by the claims below.

1. A medicinal cannabis mixture, comprising:
cannabis oil; and
one or more transdermal agents that increase permeability of skin to passage of cannabis.

2. The medicinal cannabis mixture of claim 1 wherein the one or more transdermal agents comprise one of dimethyl sulfoxide (DMSO) and Dipropylene glycol (DPG).

3. The medicinal cannabis mixture of claim 1 wherein the one or more transdermal agents comprise both dimethyl sulfoxide (DMSO) and Dipropylene glycol (DPG).

4. The medicinal cannabis mixture of claim 1 further comprising ascorbic acid as a stabilizing agent.

5. The medicinal cannabis mixture of claim 4 further comprising cholecalciferol (Vitamin D).

6. The medicinal cannabis mixture of claim 1 wherein the cannabis oil comprises one or more of tetranydrocannabinol (THC), cannabinol (CB), cannabidiol (CBD) and cannabichromene (CBC).

7. A process for administering medicinal cannabis to a subject, comprising:
preparing a mixture of cannabis oil and one or more transdermal agents that increase permeability of skin to passage of cannabis; and
applying the mixture to a subject's skin directly over either a carotid artery in the subject's neck, or directly over either or both of an ulnar or a radial artery in the subjects wrist.

8. The process for administering medicinal cannabis of claim 7, wherein the mixture of cannabis oil and one or more transdermal agents is administered by one or more drops from a syringe.

9. The process for administering medicinal cannabis of claim 7, wherein the mixture of cannabis oil and one or more transdermal agents is administered by transfer from a cotton swab carrying the mixture of cannabis oil and the one or more transdermal agents.

10. The process for administering medicinal cannabis of claim 7, wherein the mixture of cannabis oil and one or more transdermal agents is administered by application to a patch or bandage, which is then affixed to the skin of the subject.

11. The process for administering medicinal cannabis of claim 7 wherein the one or more transdermal agents comprise one of dimethyl sulfoxide (DMSO) and dipropylene glycol (DPG).

12. The process for administering medicinal cannabis of claim 7 wherein the one or more transdermal agents comprise both dimethyl sulfoxide (DMSO) and dipropylene glycol (DPG).

13. The process for administering medicinal cannabis of claim 7 further comprising ascorbic acid as a stabilizing agent.

14. The medicinal process for administering medicinal cannabis of claim 7 further comprising cholecalciferol (Vitamin D3).

15. The process for administering medicinal cannabis of claim 7 wherein the cannabis oil comprises one or more of tetranydrocannabinol (THC), cannabinol (CB), cannabidiol (CBD) and cannabichromene (CBC).
